(19) United States
(12) Patent Application Publication
(10) Pub. No.: US 2021/0283151 A1
(43) Pub. Date: Sep. 16, 2021

(54) USE OF COMPOUND IN DRUG FOR PREVENTING, TREATING, OR ALLEVIATING PAIN

(71) Applicant: ASCENTAWITS PHARMACEUTICALS, LTD., Guangdong (CN)

(72) Inventors: Jianxin Duan, Shenzhen ,Guangdong (CN); Anrong Li, Shenzhen ,Guangdong (CN); Fanying Meng, San Francisco, CA (US); Donald T Jung, Cupertino, CA (US)

(21) Appl. No.: 17/258,724

(22) PCT Filed: Apr. 26, 2019
(86) PCT No.: PCT/CN2019/084604

§ 371 Date: Jan. 7, 2021

(30) Foreign Application Priority Data
Jul. 9, 2018 (CN) 201810745871.4

(51) Int. Cl. A61K 31/675

(52) U.S. Cl. CPC A61K 31/675

(57) ABSTRACT

The present invention provides a use of a compound shown in Formula I/II in a drug for preventing, treating or alleviating pain

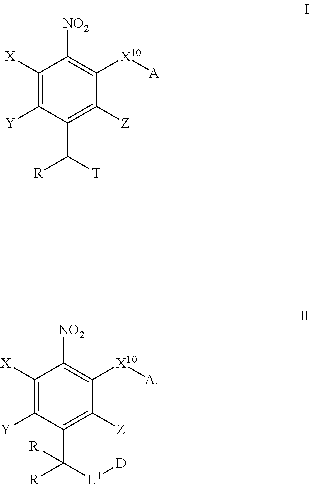

The present invention relates to use of the compounds disclosed in Patent Application No. PCT/US2016/021581, Patent Application No. PCT/US2016/025665, and Patent Application No. PCT/US2016/062114 in the manufacture of a medicament for prevention, treatment or alleviation of pain.

### TECHNICAL FIELD

### Background Art

Pain is a painful sensation caused by actual or potential tissue damage. It is both a protective response of the body and a common clinical symptom of many diseases.

The treatment of pain is very important in medicine. Traditional analgesic medicaments mainly include opioids and nonsteroidal anti-inflammatory drugs. Opioids are alkaloids extracted from opium (opium poppy) and in vitro and in vivo derivatives, which can relieve pain and produce a strong analgesic effect by interacting with specific central receptors. Nevertheless, chronic use of opioids can easily cause tolerance, dependence and addiction, and lead to such adverse reactions as respiratory inhibition and central nervous sedation. Currently, opioids are used for acute sharp pain and cancer intense pain. Nonsteroidal anti-inflammatory drugs are a class of anti-inflammatory drugs that do not contain steroid structures. They have, among others, anti-inflammatory, antirheumatic, analgesic, antipyretic and anticoagulant effects, and are widely used clinically for the relief of osteoarthritis, rheumatoid arthritis, various fevers and various pain symptoms. Nevertheless, such drugs only have moderate analgesic action and are suitable for mild and moderate chronic dull pain, but are not effective for sharp pain caused by direct stimulation of sensory endings. Additionally, they have adverse reactions such as gastrointestinal bleeding and cardiotoxicity.

Therefore, there remains a need in the art for developing new analgesic medicaments.

### SUMMARY OF THE INVENTION

In order to solve the aforementioned problems, the present invention, based on the compounds or pharmaceutically acceptable salts, or solvates thereof as disclosed in Patent Application No. PCT/US2016/021581 (WO2016/145092), Patent Application No. PCT/US2016/025665 (WO2016/161342), and Patent Application No. PCT/US2016/062114 (WO2017/087428), provides use of those compounds or pharmaceutically acceptable salts, or solvates thereof in the manufacture of a medicament for prevention, treatment or alleviation of pain.

It has been proven that the compounds disclosed in the patent applications PCT/US2016/021581, PCT/US2016/025665 and PCT/US2016/062114, as internationally original, small molecule targeted therapeutic drugs with high tumor selectivity, have shown excellent anticancer effects in various preclinical cells and animal models. These compounds, as specific substrates of the aldo-keto reductase AKR1C3, can be quickly and effectively reduced only in cancer cells which overexpress AKR1C3, thereby releasing cytotoxins to result in highly selective cancer cell killing effects.

The research group contemplates that since the compounds disclosed in the aforementioned invention patent applications are used as specific substrates of the aldo-keto reductase AKR1C3 (hereinafter referred to as specific substrates), they are actually anticancer alkylating agent prodrugs, which are specifically activated under the action of the aldo-keto reductase AKR1C3 and are metabolized to produce a cytotoxic alkylating agent. Take AST-2870 (the compound TH-2870 disclosed in the aforementioned PCT/US2016/021581) as an example.

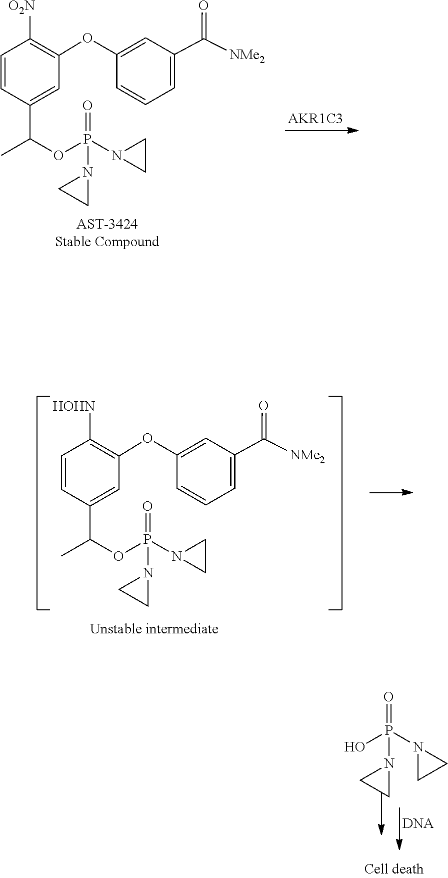

Evidently, the aldo-keto reductase AKR1C3 needs to bind to the specific substrates.

According to the research literature (Samad T A, Moore K A, Sapirstein A, et al. Interleukin-1 [beta]-mediated induction of Cox-2 in the CNS contributes to inflammatory pain hypersensitivity[J]. Nature, 2001, 410(6827):471-5.; Baojian Z, Yanbing Y, Gele A, et al. Tanshinone IIA Attenuates Diabetic Peripheral Neuropathic Pain in Experimental Rats via Inhibiting Inflammation[J]. Evidence-Based Complementary and Alternative Medicine, 2018, 2018:1-8.; Lovering A, Ride J, Bunce C, et al. Crystal Structures of Prostaglandin D₂ 11-Ketoreductase (AKR1C3) in Complex with the Nonsteroidal Anti-Inflammatory Drugs Flufenamic Acid and Indomethacin[J]. Cancer Research, 2004, 64(5):1802-1810.; Matsuura K, Shiraishi H, Hara A, et al. Identification of a principal mRNA species for human 3alpha-hydroxysteroid dehydrogenase isoform (AKR1C3) that exhibits high prostaglandin D₂ 11-ketoreductase activity.[J]. Journal of Biochemistry, 1998, 124(5):940-6.), it is known that the aldo-keto reductase AKR1C3 plays an important catalytic role in the biochemical pathway of the conversion of prostaglandin H2/D2 to prostaglandin E2/F2:

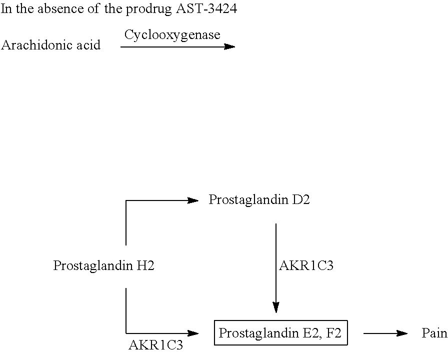

The inventors contemplate that since the specific substrates can bind to the aldo-keto reductase AKR1C3, can it be an inhibitor of the aldo-keto reductase AKR1C3, thereby reducing the level of prostaglandin E2/F2, and preventing, treating or alleviating the pain caused by cancer or inflammation?

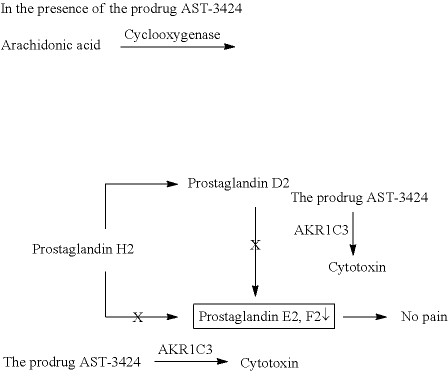

Regarding above assumptions, the inventors have designed experiments verifying that the aforementioned specific substrates, namely DNA alkylating agent targeting overexpressed aldo-keto reductase AKR1C3, can inhibit the activity of the aldo-keto reductase AKR1C3. Moreover, the animal experiment proves that the specific substrates can reduce the content of prostaglandin E2/F2 in the blood. Accordingly, it can be proved that the aforementioned specific substrates have an analgesic effect.

Regarding the analgesic effect of the aforementioned compounds, especially the use thereof in the manufacture of a medicament for preventing, treating or alleviating the pain caused by cancer or inflammation, the present invention provides the following technical solutions.

In one aspect, the present invention provides use of a compound of formula I or a pharmaceutically acceptable salt, or a solvate thereof in the manufacture of a medicament for preventing, treating or alleviating pain,

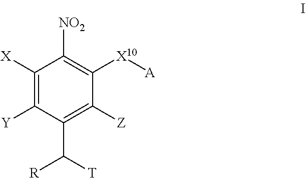

wherein

X¹⁰ is O, S, SO, or SO₂;

A is C₆-C₁₀ aryl or substituted aryl, 5-15 membered heteroaryl or substituted heteroaryl, or —N═CR¹R², wherein each R¹ and R² independently is hydrogen, C₁-C₆ alkyl, C₃-C₈ cycloalkyl, C₆-C₁₀ aryl, 4-15 membered heterocycle, 5-15 membered heteroaryl, ether, —CONR¹³R¹⁴, or —NR¹³COR¹⁴;

each X, Y, and Z independently is hydrogen, CN, halo, C₁-C₆ alkyl, C₂-C₆ alkenyl, C₂-C₆ alkynyl, C₃-C₈ cycloalkyl, C₆-C₁₀ aryl, 4-15 membered heterocycle, 5-15 membered heteroaryl, ether, —CONR¹³R¹⁴, or —NR¹³COR¹⁴;

R is hydrogen, C₁-C₆ alkyl, C₂-C₆ alkenyl, C₂-C₆ alkynyl, C₃-C₈ cycloalkyl, C₆-C₁₀ aryl, 4-15 membered heterocycle, 5-15 membered heteroaryl, ether, —CONR¹³R¹⁴, or —NR¹³COR¹⁴;

each R¹³ and R¹⁴ independently is hydrogen, C₁-C₆ alkyl, C₃-C₈ cycloalkyl, C₆-C₁₀ aryl, 4-15 membered heterocycle, 5-15 membered heteroaryl or ether, or R¹³ and R¹⁴ together with the nitrogen atom to which they are bonded to form 5-7 membered heterocyclyl group;

T comprises a phosphoramidate alkylating agent; and

wherein the alkyl, alkenyl, alkynyl, cycloalkyl, aryl, heterocycle, heteroaryl, ether groups are optionally substituted.

Preferably, the compound is of formula I-A,

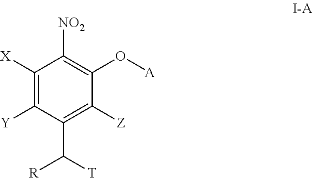

wherein the remaining variables are defined as above.

Alternatively, X¹⁰ is S.

More preferably, in the compound, T is OP(Z¹)(NR³⁰CH₂CH₂X¹)₂, OP(Z¹)(NR³⁰₂)(N(CH₂CH₂X¹)₂), OP(Z¹)(N(CH₂)₂)₂ or OP(Z¹)(N(CH₂CH₂X¹)₂)₂, wherein each R³⁰ independently is hydrogen or C₁-C₆ alkyl or two R³⁰ groups together with the nitrogen atom to which they are bonded to form 5-7 membered heterocyclyl group, Z¹ is O or S, and X¹ is Cl, Br, or OMs or other leaving groups.

Further preferably, in the compound, T is OP(Z¹)(NHCH₂CH₂Cl)₂, OP(Z¹)(NHCH₂CH₂Br)₂, OP(Z¹)(NH₂)(N(CH₂CH₂X¹)₂), OP(Z¹)(N(CH₂)₂)₂ or OP(Z¹)(N(CH₂CH₂Cl)₂)₂, wherein Z¹ is O or S, and X¹ is Cl, Br, or OMs.

According to particular embodiments of the invention, in the compound, Z¹ is O or S.

According to particular embodiments of the invention, in the compound, T is OP(O)(N(CH₂CH₂))₂, OP(O)(NHCH₂CH₂Cl)₂, OP(O)(NHCH₂CH₂Br)₂ or OP(O)(NH₂)(N(CH₂CH₂Cl)₂).

According to particular embodiments of the invention, in the compound, Z is hydrogen.

According to particular embodiments of the invention, in the compound, X is hydrogen.

According to particular embodiments of the invention, in the compound, Y is hydrogen or halo.

Preferably, in the compound, A is optionally substituted C₆-C₁₀ aryl.

More preferably, in the compound, A is optionally substituted phenyl.

Preferably, in the compound, A is optionally substituted 5-15 membered heteroaryl.

More preferably, in the compound, A is optionally substituted pyridyl.

More preferably, in the compound, A is —N═CR¹R² where R¹ and R² are defined as above.

According to particular embodiments of the invention, in the compound, R is hydrogen.

Preferably, in the compound, R is C₁-C₆ alkyl.

According to particular embodiments of the invention, in the compound, R is methyl. Regarding the use described herein, the compounds include individual diastereomers and other geometric isomers, and enantiomers, and mixtures of enantiomers, diastereomers, and geometric isomers other than diastereomers.

The compound of formula I described above in the present patent is disclosed in PCT Application No. PCT/US2016/021581 (International Publication No. WO2016/145092), which corresponds to Chinese Patent Application No. CN201680015078.8 (Publication No. CN107530556A), the disclosure of which is incorporated herein by reference in its entirety. In this PCT application, the general structure of the compound, the radical substitution, the preferred general structure, the preferred specific compounds and the like are all described in detail, and the entire contents thereof are incorporated herein by reference. Therefore, the use of the compound as described above or the pharmaceutically acceptable salt, or the solvate thereof in the manufacture of a medicament for the prevention, treatment or alleviation of pain in the present invention covers all the contents about the compound or the pharmaceutically acceptable salt, or the solvate thereof as provided in PCT Application No. PCT/US2016/021581.

In another aspect, the invention provides use of a compound of formula II or a pharmaceutically acceptable salt, or a solvate thereof in the manufacture of a medicament for the prevention, treatment or alleviation of pain,

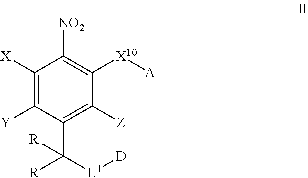

wherein

X¹⁰ is O, S, SO, or SO₂;

A is C₆-C₁₀ aryl or substituted aryl, 5-15 membered heteroaryl or substituted heteroaryl, or —N═CR¹R², wherein each R¹ and R² independently is hydrogen, C₁-C₆ alkyl, C₃-C₈ cycloalkyl, C₆-C₁₀ aryl, 4-15 membered heterocycle, 5-15 membered heteroaryl, ether, —CONR¹³R¹⁴ or —NR¹³COR¹⁴;

each X, Y, and Z independently is hydrogen, CN, halo, C₁-C₆ alkyl, C₂-C₆ alkenyl, C₂-C₆ alkynyl, C₃-C₈ cycloalkyl, C₆-C₁₀ aryl, 4-15 membered heterocycle, 5-15 membered heteroaryl, ether, —CONR¹³R¹⁴, or —NR¹³COR¹⁴;

each R independently is hydrogen, C₁-C₆ alkyl, C₂-C₆ alkenyl, C₂-C₆ alkynyl, C₃-C₈ cycloalkyl, C₆-C₁₀ aryl, 4-15 membered heterocycle, 5-15 membered heteroaryl, ether, —CONR¹³R¹⁴, or —NR¹³COR¹⁴;

each R¹³ and R¹⁴ independently is hydrogen, C₁-C₆ alkyl, C₃-C₈ cycloalkyl, C₆-C₁₀ aryl, 4-15 membered heterocycle, 5-15 membered heteroaryl, or ether, or R¹³ and R¹⁴ together with the nitrogen atom to which they are bonded to form 5-7 membered heterocyclyl group;

L¹ and D are defined as follows:

L¹ is selected from:

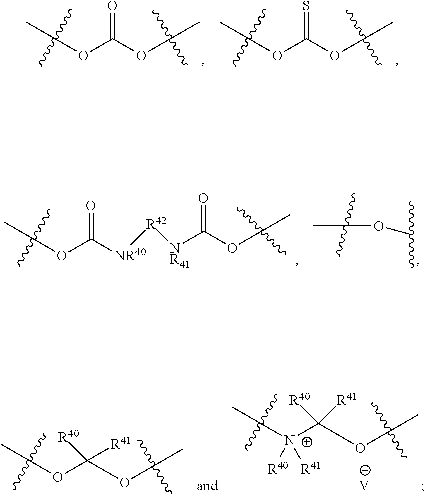

wherein R⁴⁰ and R⁴¹ are independently hydrogen, C₁-C₆ alkyl, C₂-C₆ alkenyl, C₂-C₆ alkynyl, C₃-C₈ cycloalkyl, C₆-C₁₀ aryl, 4-15 membered heterocycle, or 5-15 membered heteroaryl; R⁴² is C₂-C₃ alkylene or heteroalkylene optionally substituted with 1-3 C₁-C₆ alkyl, V(−) is any anion, preferably, a pharmaceutically acceptable anion, and

D is a moiety such that D-OH is an anticancer drug wherein OH is an aliphatic or a phenolic hydroxy group or is an OH moiety attached to a phosphorous atom as provided herein; in other words, D is the remaining group in the anticancer drug D-OH after the hydroxyl group is removed therefrom;

alternatively

L¹ is:

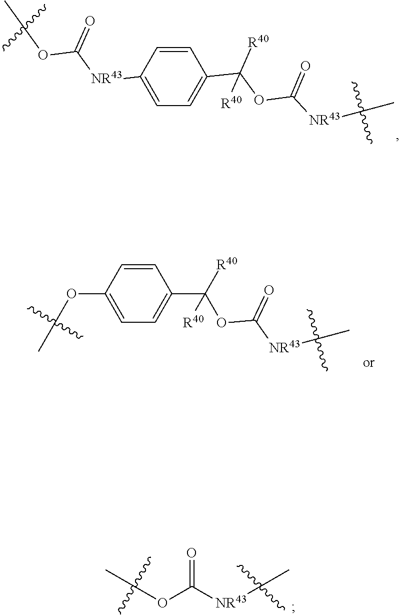

wherein R⁴⁰ is defined as above, R⁴³ is hydrogen or together with D forms a heterocycle, and the phenyl moiety is optionally substituted, and

D is a moiety such that D-NR⁴³H is an anticancer drug; in other words, D is the remaining group in the anticancer drug D-NR⁴³H after amino or amine is removed therefrom;

alternatively

L¹ is a bond, —O—C(R⁴⁰R⁴¹)₂—, —O—C(R⁴⁰R⁴¹)—NR⁴⁰R⁴¹(+)—C(R⁴⁰R⁴¹)— or

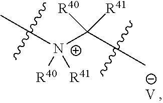

wherein R⁴⁰, R⁴¹ and V are defined as above, and

D is an anticancer drug containing a primary or a secondary amine, wherein the primary or the secondary amine is bonded to L¹; and

wherein the alkyl, alkenyl, alkynyl, cycloalkyl, aryl, heterocycle, heteroaryl, and ether groups are optionally substituted.

Preferably, the compound is of formula II-A,

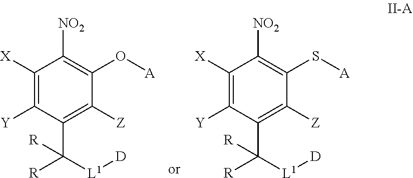

wherein the remaining variables are defined as above.

In another example, X¹⁰ is S.

More preferably, the compound is of formula IIA-1:

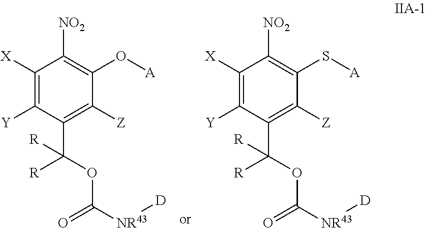

wherein the remaining variables are defined as above, and D is a moiety of the cytotoxic agent HNR⁴³-D containing a primary or a secondary amine group; in other words, D is the remaining group in the cytotoxic agent HNR⁴³-D after the primary or a secondary amine group is removed therefrom.

Alternatively, more preferably, the compound is of formula III-2 or IIIA-3:

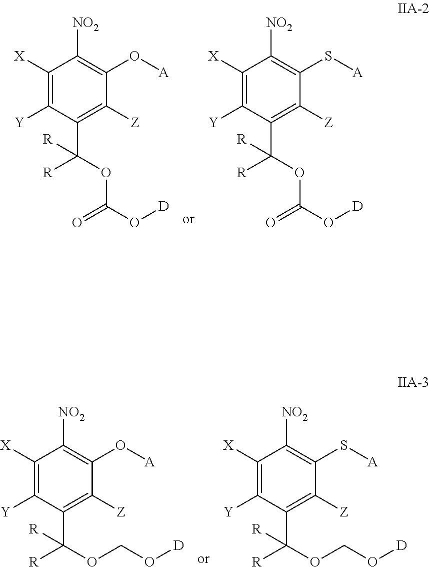

wherein the remaining variables are defined as above, and D is a moiety of the cytotoxic agent HO-D containing at least one hydroxyl group; in other words, D is the remaining group in the cytotoxic agent HO-D after the hydroxyl group is removed therefrom.

Alternatively, more preferably, the compound is of IIA-4, IIA-6 or IIA-6-i:

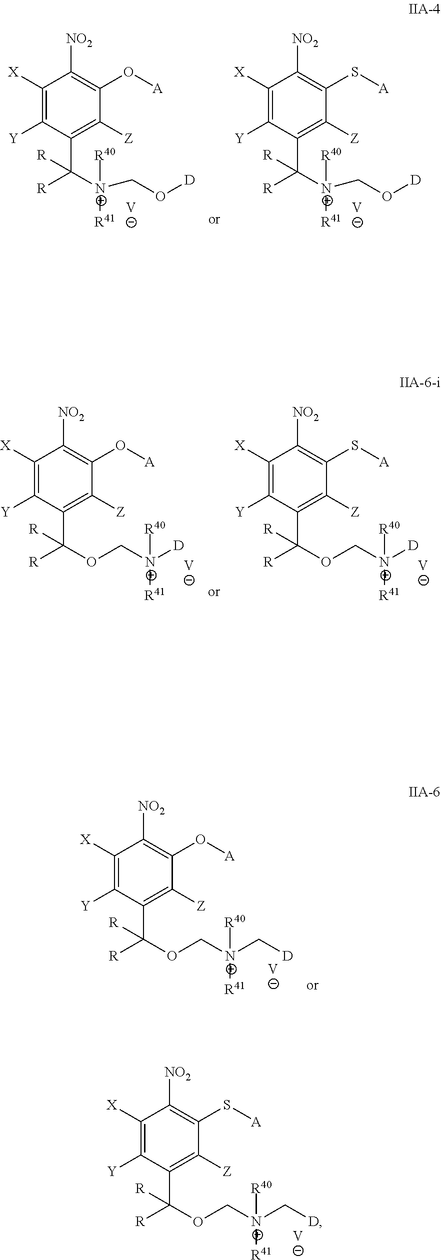

wherein the variables are defined as above; wherein in IIA-4, HO-D is cytotoxic agent containing at least one hydroxyl group; in other words, D is a moiety of the cytotoxic agent HO-D containing at least one hydroxyl group, or D is the remaining group in the cytotoxic agent HO-D after the hydroxyl group(s) is/are removed therefrom; in IIA-6-i, DNR⁴⁰R⁴¹ is a drug; in other words, D is a moiety such that DNR⁴⁰R⁴¹ is an anticancer drug or D is the remaining moiety in the anticancer DNR⁴⁰R⁴¹ after NR⁴⁰R⁴¹ is removed therefrom; and in IIA-6, D is a drug containing a secondary amine, wherein the secondary amine is bonded to the methylene group; in other words, D is a drug containing a secondary amine, and is bonded via the secondary amine contained therein to the methylene group so as to be linked to —NR⁴⁰R⁴¹ as shown above.

Alternatively, more preferably, the compound is of IIA-5 or IIA-7:

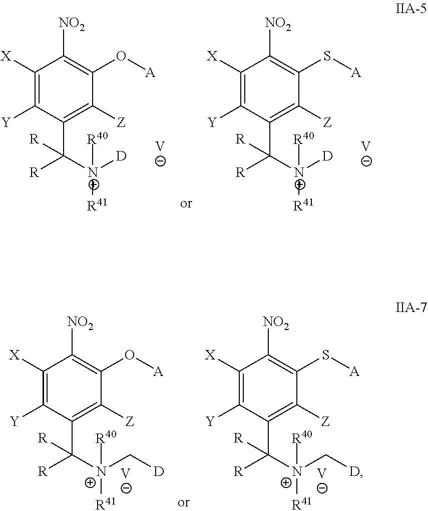

wherein the variables are defined as above; wherein in IIA-5, DNR⁴⁰R⁴¹ is a drug; in other words, D is a moiety such that DNR⁴⁰R⁴¹ is an anticancer drug or D is the remaining moiety in the anticancer DNR⁴⁰R⁴¹ after NR⁴⁰R⁴¹ is removed therefrom; and in IIA-7, D is a drug containing a secondary amine, wherein the secondary amine is bonded to the methylene group as shown above.

According to particular embodiments of the invention, in the compound, Z is hydrogen.

According to particular embodiments of the invention, in the compound, X is hydrogen.

According to particular embodiments of the invention, in the compound, Y is hydrogen or halo.

Preferably, in the compound, A is optionally substituted C₆-C₁₀ aryl.

More preferably, in the compound, A is optionally substituted phenyl.

Preferably, in the compound, A is optionally substituted 5-15 membered heteroaryl.

More preferably, in the compound, A is optionally substituted pyridyl.

More preferably, in the compound, A is —N═CR¹R², wherein R¹ and R² are defined as above.

According to particular embodiments of the invention, in the compound, each R is hydrogen.

Preferably, in the compound, one of the R groups is hydrogen and the other R group is C₁-C₆ alkyl, or both R groups are non-hydrogen substituents as defined above.

According to particular embodiments of the invention, in the compound, R is methyl.

Preferably, in the compound, each of R⁴⁰, R⁴¹ and R⁴³ is independently hydrogen or methyl and R⁴² is —CH₂—CH₂— or CH₂—C(Me)₂-.

Particularly, as used herein, D excludes a phosphoramidate alkylating agent such as —P(Z¹)(NR³⁰CH₂CH₂X¹)₂, —P(Z¹)(NR³⁰₂)(N(CH₂CH₂X¹)₂), —P(Z¹)(N(CH₂CH₂))₂ or —P(Z¹)(N(CH₂CH₂X¹)₂)₂, wherein each R³⁰ independently is hydrogen or C₁-C₆ alkyl or two R³⁰ groups together with the nitrogen atom to which they are bonded to form 5-7 membered heterocyclyl group, Z¹ is O or S, and X¹ is Cl, Br, or OMs or another leaving group.

Regarding the use described herein, the compounds include individual diastereomers and other geometric isomers, and enantiomers, and mixtures of enantiomers, diastereomers, and geometric isomers other than diastereomers.

The compound of formula II described above in the present patent is disclosed in PCT Application No. PCT/US2016/025665 (International Publication No. WO2016/161342), which corresponds to Chinese Patent Application No. CN201680020013.2 (Publication No. CN108136214A), the disclosure of which is incorporated herein by reference in its entirety. In this PCT application, the general structure of the compound, the radical substitution, the preferred general structure, the preferred specific compounds and the like are all described in detail, and the entire contents thereof are incorporated herein by reference. Therefore, the use of the compound as described above or the pharmaceutically acceptable salt, or the solvate thereof in the present invention in the manufacture of a medicament for the prevention, treatment or alleviation of pain covers all the contents about the compound or the pharmaceutically acceptable salt, or the solvate thereof as provided in PCT Application No. PCT/US2016/025665.

According to particular embodiments of the invention, the compound I is the compound AST-3424 as shown below:

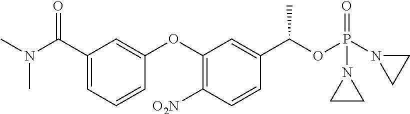 [SMILES: C[C@H](OP(=O)(N1CC1)N1CC1)c1ccc([N+](=O)[O-])c(Oc2cccc(C(=O)N(C)C)c2)c1]

AST-3424 (The S-isomer of compound 2870 in the three aforementioned PCT applications)

The compounds for the use described herein may also be used in the form of their salts. In other words, the present invention provides use of pharmaceutically acceptable salts of the compounds as shown herein in the manufacture of a medicament for the prevention, treatment or alleviation of pain. Herein, the salts may be basic salts, including the salts of the compounds with an inorganic base (such as alkali metal hydroxide and alkaline earth metal hydroxide) or with an organic base (such as monoethanolamine, diethanolamine or triethanolamine). Alternatively, the salts may be acid salts, including the salts of the compounds with an inorganic acid (such as hydrochloric acid, hydrobromic acid, hydroiodic acid, nitric acid, perchloric acid, sulfuric acid or phosphoric acid) or with an organic acid (such as methanesulfonic acid, trifluoromethanesulfonic acid, ethanesulfonic acid, benzenesulfonic acid, p-toluenesulfonic acid, fumaric acid, oxalic acid, maleic acid and citric acid). It is a well-known technology in the art to select and prepare acceptable salts, solvates, and the like of a compound.

The compounds for the use described herein may also be used in the form of solvates. In other words, the present invention provides use of pharmaceutically acceptable solvates of the compounds as shown herein in the manufacture of a medicament for the prevention, treatment or alleviation of pain. Therein, the solvate is hydrate, alcoholate and the like.

Regarding the use described herein, the pain is caused by cancer which includes, but is not limited to lung cancer, non-small cell lung cancer, liver cancer, pancreatic cancer, stomach cancer, bone cancer, esophagus cancer, breast cancer, prostate cancer, testicular cancer, colon cancer, ovarian cancer, bladder cancer, cervical cancer, melanoma, squamous cell carcinoma, basal cell carcinoma, adenocarcinoma, sweat gland carcinoma, sebaceous gland carcinoma, papillary carcinoma, papillary adenocarcinoma, adenocarcinoma cystic, cystic carcinoma, medullary carcinoma, bronchial carcinoma, osteocyte carcinoma, epithelial carcinoma, carcinoma of bile duct, choriocarcinoma, embryonal carcinoma, seminoma, Wilm's tumor, glioblastoma, astrocytoma, medulloblastoma, craniopharyngioma, ependymoma, pineal tumor, hemocytoblastoma, vocal cords neuroma, meningioma, neuroblastoma, optic neuroblastoma, retinoblastoma, neurofibroma, fibrosarcoma, fibroblastoma, fibroma, fibroadenoma, fibrochondroma, fibrocystoma, fibromyxoma, fibroosteoma, fibromyxosarcoma, fibropapilloma, myxos arcoma, myxocystoma, myxochondroma, myxochondrosarcoma, myxochondrofibrosarcoma, myxadenoma, myxoblastoma, liposarcoma, lipoma, lipoadenoma, lipoblastoma, lipochondroma, lipofibroma, lipoangioma, myxolipoma, chondrosarcoma, chondroma, chondromyoma, chordoma, choriocarcinoma, chorioepithelioma, chorioblastoma, osteosarcoma, osteoblastoma, osteochondrofibroma, osteochondrosarcoma, osteochondroma, osteocystoma, osteodentinoma, osteofibroma, fibrosarcoma of bone, angiosarcoma, hemangioma, angiolipoma, angiochondroma, hemangioblastoma, angiokeratoma, angioglioma, angioendothelioma, angiofibroma, angiomyoma, angiolipoma, angiolymphangioma, angiolipoleiomyoma, angiomyolipoma, angiomyoneuroma, angiomyxoma, angioreticuloma, lymphangiosarcoma, lymphogranuloma, lymphangioma, lymphoma, lymphomyxoma, lymphosarcoma, lymphangiofibroma, lymphocytoma, lymphoepithelioma, lymphoblastoma, endothelioma, endoblastoma, synovioma, synovial sarcoma, mesothelioma, connective tissue tumor, Ewing's tumor, leiomyoma, leiomyosarcoma, leiomyoblastoma, leiomyofibroma, rhabdomyoma, rhabdomyosarcoma, rhabdomyomyxoma, acute lymphatic leukemia, acute myelogenous leukemia, chronic disease cells, polycythemia, lymphoma, endometrial cancer, glioma, colorectal cancer, thyroid cancer, urothelial cancer or multiple myeloma.

From the above experiments conducted by the inventor of the present application and the relevant research literature, it can be obviously known that regarding the use described herein, the pain is caused by inflammation.

Regarding the use described herein, the prepared medicament may further include other compounds or drugs used in combination with the compounds shown herein. The other compounds or drugs can also be used for the prevention, treatment or alleviation of pain.

Regarding the use described herein, the prepared medicament contains a specific dosage range of the shown compounds or salts or solvates thereof, and/or the prepared medicament is in a specific dosage form and is administered using a specific mode of administration.

The dosage of the medicament used for prevention, treatment or alleviation of pain, or the dosage of the compounds or salts or solvates thereof contained in the medicament usually depends on the specific compound applied, the patient, the specific disease or condition and the severity thereof, the route and frequency of administration and the like, and needs to be determined by the attending physician according to specific conditions. For example, when the compound or medicament provided by the present invention is administered by the oral route, the dosage may be 0.1 to 30 mg/7 days, preferably 1 to 10 mg/7 days, more preferably 5 mg/day; the dosage may be administered once every 7 days or divided into two dosages for administration twice every 7 days; preferably, the dosage is administered once every 7 days.

Regarding the use described herein, the prepared medicament may also contain pharmaceutically acceptable auxiliaries or excipients. The medicament can be any dosage form for clinical administration, such as tablets, suppositories, dispersible tablets, enteric-coated tablets, chewable tablets, orally disintegrating tablets, capsules, sugar coated agents, granules, dry powders, oral solutions, a small needle for injection, lyophilized powder for injection, or infusion solutions. According to the specific dosage form and the mode of administration, the pharmaceutically acceptable auxiliaries or excipients in the medicament may include one or more of the following: diluent, solubilizer, disintegrant, suspension, lubricant, adhesive, filler, flavoring agent, sweetener, antioxidant, surfactant, preservative, wrapping agent and pigment.

Based on the use provided in the present invention, the present invention also relates to a method for prevention, treatment or alleviation of pain, the method comprising administering an preventively, therapeutically and alleviatively effective amount of the compound of the present invention or a pharmaceutically acceptable salt or a solvate thereof to a subject in need thereof. Preferably, the subject is a mammal, more preferably a human. The pain is caused by cancer which includes, but is not limited to lung cancer, non-small cell lung cancer, liver cancer, pancreatic cancer, stomach cancer, bone cancer, esophagus cancer, breast cancer, prostate cancer, testicular cancer, colon cancer, ovarian cancer, bladder cancer, cervical cancer, melanoma, squamous cell carcinoma, basal cell carcinoma, adenocarcinoma, sweat gland carcinoma, sebaceous gland carcinoma, papillary carcinoma, papillary adenocarcinoma, adenocarcinoma cystic, cystic carcinoma, medullary carcinoma, bronchial carcinoma, osteocyte carcinoma, epithelial carcinoma, carcinoma of bile duct, choriocarcinoma, embryonal carcinoma, seminoma, Wilm's tumor, glioblastoma, astrocytoma, medulloblastoma, craniopharyngioma, ependymoma, pineal tumor, hemocytoblastoma, vocal cords neuroma, meningioma, neuroblastoma, optic neuroblastoma, retinoblastoma, neurofibroma, fibrosarcoma, fibroblastoma, fibroma, fibroadenoma, fibrochondroma, fibrocystoma, fibromyxoma, fibroosteoma, fibromyxosarcoma, fibropapilloma, myxosarcoma, myxocystoma, myxochondroma, myxochondrosarcoma, myxochondrofibrosarcoma, myxadenoma, myxoblastoma, liposarcoma, lipoma, lipoadenoma, lipoblastoma, lipochondroma, lipofibroma, lipoangioma, myxolipoma, chondrosarcoma, chondroma, chondromyoma, chordoma, choriocarcinoma, chorioepithelioma, chorioblastoma, osteosarcoma, osteoblastoma, osteochondrofibroma, osteochondrosarcoma, osteochondroma, osteocystoma, osteodentinoma, osteofibroma, fibrosarcoma of bone, angiosarcoma, hemangioma, angiolipoma, angiochondroma, hemangioblastoma, angiokeratoma, angioglioma, angioendothelioma, angiofibroma, angiomyoma, angiolipoma, angiolymphangi oma, angiolipoleiomyoma, angiomyolipoma, angi omyoneuroma, angiomyxoma, angioreticuloma, lymphangiosarcoma, lymphogranuloma, lymphangioma, lymphoma, lymphomyxoma, lymphosarcoma, lymphangiofibroma, lymphocytoma, lymphoepithelioma, lymphoblastoma, endothelioma, endoblastoma, synovioma, synovial sarcoma, mesothelioma, connective tissue tumor, Ewing's tumor, leiomyoma, leiomyosarcoma, leiomyoblastoma, leiomyofibroma, rhabdomyoma, rhabdomyosarcoma, rhabdomyomyxoma, acute lymphatic leukemia, acute myelogenous leukemia, chronic disease cells, polycythemia, lymphoma, endometrial cancer, glioma, colorectal cancer, thyroid cancer, urothelial cancer or multiple myeloma.

Based on the use provided in the present invention, the present invention further provides a medicament comprising the compound of formula I or formula II as defined above useful for prevention, treatment or alleviation of pain caused by cancer or inflammation.

Based on the use provided in the present invention, the present invention further provides a method for treating pain caused by cancer or inflammation, comprising a step of administering the aforementioned medicament; and a step for measuring the content of the reductase AKR1C3 of cancer cells in a patient using AKR1C3 antibodies (where the content of the AKR1C3 reductase is measured to be equal to or greater than the predetermined value, the aforementioned medicament is administered to the patient).

### BRIEF DESCRIPTION OF DRAWINGS

FIG. 1 depicts the experiment showing the effect of application of AST-3424 on the production of reduced progesterone.

### DETAILED DESCRIPTION OF THE INVENTION

The present invention will be described below with reference to specific examples. Those skilled in the art could understand that these examples are only used for describing the invention and do not in any way limit its scope.

The experimental methods in the following examples are all conventional methods unless otherwise specified. The raw materials of the medicaments, the reagents and the like used in the following examples are all commercially available products unless otherwise specified.

The compounds shown in the present invention and the methods for synthesizing the same are disclosed in Patent Application No. PCT/US2016/021581 (WO2016/145092), Patent Application No. PCT/US2016/025665 (WO2016/161342), and Patent Application No. PCT/US2016/062114 (WO2017/087428).

The following definitions are provided to assist the reader. Unless otherwise defined, all terms of art, notations, and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not be construed as representing a substantial difference over the definition of the term as generally understood in the art.

All numerical designations, e.g., pH, temperature, time, concentration, and weight, including ranges of each thereof, are approximations that typically may be varied (+) or (−) by increments of 0.1, 1.0, or 10.0, as appropriate. All numerical designations may be understood as preceded by the term “about”. Reagents described herein are exemplary and equivalents of such may be known in the art.

“Cₓ-C_{y}” or “C_{x-y}” before a group refers to a range of the number of carbon atoms that are present in that group. For example, C₁-C₆ alkyl refers to an alkyl group having at least 1 and up to 6 carbon atoms.

“Alkoxy” refers to —O-Alkyl.

“Amino” refers to NR^{p}R^{q} wherein R^{p} and R^{q} independently are hydrogen or C₁-C₆ alkyl, or R^{p} and R^{q} together with the nitrogen atom to which they are bonded to form a 4-15 membered heterocycle.

“Aryl” refers to an aromatic group of from 6 to 14 carbon atoms and no ring heteroatoms and having a single ring (e.g., phenyl) or multiple condensed (fused) rings (e.g., naphthyl or anthryl). For multiple ring systems, including fused, bridged, and spiro ring systems having aromatic and non-aromatic rings that have no ring heteroatoms, the term “aryl” or “Ar” applies when the point of attachment is at an aromatic carbon atom (e.g., 5,6,7,8 tetrahydronaphthalene-2-yl is an aryl group as its point of attachment is at the 2-position of the aromatic phenyl ring).

According to specific embodiments of the present application, C₆-C₁₀ aryl can be phenyl, naphthyl and various substituted phenyl or naphthyl.

“Heteroaryl” refers to an aromatic group of from 1 to 14 carbon atoms and 1 to 6 heteroatoms selected from the group consisting of oxygen, nitrogen, and sulfur and includes single ring (e.g. imidazolyl-2-yl and imidazol-5-yl) and multiple ring systems (e.g. imidazopyridyl, benzotriazolyl, benzimidazol-2-yl and benzimidazol-6-yl). For multiple ring systems, including fused, bridged, and spiro ring systems having aromatic and non-aromatic rings, the term “heteroaryl” applies if there is at least one ring heteroatom, and the point of attachment is at an atom of an aromatic ring (e.g. 1,2,3,4-tetrahydroquinolin-6-yl and 5,6,7,8-tetrahydroquinolin-3-yl). In some embodiments, the nitrogen and/or the sulfur ring atom(s) of the heteroaryl group are optionally oxidized to provide for the N-oxide (N→O), sulfinyl, or sulfonyl moieties. The term heteroaryl or 5-15 membered heteroaryl includes, but is not limited to, acridinyl, azocinyl, benzimidazolyl, benzofuranyl, benzothiofuranyl, benzothiophenyl, benzoxazolyl, benzothiazolyl, benzotriazolyl, benzotetrazolyl, benzisoxazolyl, benzisothiazolyl, benzothienyl, benzimidazolinyl, carbazolyl, NH-carbazolyl, carbolinyl, chromanyl, chromenyl, cinnolinyl, dithiazinyl, furanyl, furazanyl, imidazolidinyl, imidazolinyl, imidazopyridyl, imidazolyl, indazolyl, indolenyl, indolinyl, indolizinyl, indolyl, isobenzofuranyl, isochromanyl, isoindazolyl, isoindolinyl, isoindolyl, isoquinolinyl, isoquinolyl, isothiazolyl, isoxazolyl, naphthyridinyl, octahydroisoquinolinyl, oxadiazolyl, oxazolidinyl, oxazolyl, pyrimidinyl, phenanthridinyl, phenanthrolinyl, phenazinyl, phenothiazinyl, phenoxathiinyl, phenoxazinyl, phthalazinyl, piperazinyl, pteridinyl, purinyl, pyranyl, pyrazinyl, pyrazolidinyl, pyrazolinyl, pyrazolyl, pyridazinyl, pyridooxazolyl, pyridoimidazolyl, pyridothiazole, pyridinyl, pyridyl, pyrimidinyl, pyrrolyl, quinazolinyl, quinolinyl, quinoxalinyl, quinuclidinyl, tetrahydroisoquinolinyl, tetrahydroquinolinyl, tetrazolyl, thiadiazinyl, thiadiazolyl, thianthrenyl, thiazolyl, thienyl, thienothiazolyl, thienooxazolyl, thienoimidazolyl, thiophenyl, triazinyl and xanthenyl.

“Alkyl” refers to monovalent saturated aliphatic hydrocarbyl groups having from 1 to 10 carbon atoms and, in some embodiments, from 1 to 6 carbon atoms. “C_{x-y} alkyl” refers to alkyl groups having from x to y carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), tert-butyl ((CH₃)₃C—), n-pentyl (CH₃CH₂CH₂CH₂CH₂—), and neopentyl ((CH₃)₃CCH₂—).

“Cycloalkyl” refers to a saturated or partially saturated cyclic group of from 3 to 14 carbon atoms and no ring heteroatoms and having a single ring or multiple rings including fused, bridged, and spiro ring systems. For multiple ring systems having aromatic and non-aromatic rings that have no ring heteroatoms, the term “cycloalkyl” applies when the point of attachment is at a non-aromatic carbon atom (e.g. 5,6,7,8-tetrahydronaphthalene-5-yl). The term “cycloalkyl” or “C₃-C₈ cycloalkyl” includes cycloalkenyl groups. Examples of cycloalkyl groups or C₃-C₈ cycloalkyl groups include, for instance, adamantyl, cyclopropyl, cyclobutyl, cyclopentyl, cyclooctyl, and cyclohexenyl.

“Heterocyclic” or “heterocycle” or “heterocycloalkyl” or “heterocyclyl” refers to a saturated or partially saturated cyclic group having from 1 to 14 carbon atoms and from 1 to 6 heteroatoms selected from the group consisting of nitrogen, sulfur, or oxygen and includes single ring and multiple ring systems including fused, bridged, and spiro ring systems. For multiple ring systems having aromatic and/or non-aromatic rings, the terms “heterocyclic”, “heterocycle”, “heterocycloalkyl”, or “heterocyclyl” apply when there is at least one ring heteroatom, and the point of attachment is at an atom of a non-aromatic ring (e.g. 1,2,3,4-tetrahydroquinoline-3-yl, 5,6,7,8-tetrahydroquinoline-6-yl, and decahydroquinolin-6-yl). In some embodiments, the heterocyclic groups herein are 3-15 membered, 4-14 membered, 5-13 membered, 7-12 membered, or 5-7 membered heterocycles. In some other embodiments, the heterocycles contain 4 heteroatoms. In some other embodiments, the heterocycles contain 3 heteroatoms. In another embodiment, the heterocycles contain up to 2 heteroatoms. In some embodiments, the nitrogen and/or sulfur atom(s) of the heterocyclic group are optionally oxidized to provide for the N-oxide, sulfinyl, sulfonyl moieties. Heterocyclyl includes, but is not limited to, tetrahydropyranyl, piperidinyl, N-methylpiperidin-3-yl, piperazinyl, N-methylpyrrolidin-3-yl, 3-pyrrolidinyl, 2-pyrrolidon-1-yl, morpholinyl, and pyrrolidinyl. A prefix indicating the number of carbon atoms (e.g., C₃₋₁₀) refers to the total number of carbon atoms in the portion of the heterocyclyl group exclusive of the number of heteroatoms. A divalent heterocyclic radical will have the appropriately adjusted hydrogen content.

“Ether” refers to a C₁-C₆ alkyl group substituted with 1-3 C₁-C₆ alkoxy groups, wherein alkoxy refers to —O-alkyl.

“Halo” refers to one or more of fluoro, chloro, bromo, and iodo.

“Alkenyl” refers to a linear or branched hydrocarbyl group having from 2 to 10 carbon atoms and in some embodiments from 2 to 6 carbon atoms or 2 to 4 carbon atoms and having at least 1 site of vinyl unsaturation (>C=—). For example, C_{x-y} alkenyl refers to alkenyl groups having from x to y carbon atoms and is meant to include, for example, ethenyl, propenyl, 1,3-butadienyl, and the like.

“Alkynyl” refers to a linear monovalent hydrocarbon radical or a branched monovalent hydrocarbon radical having from 2 to 10 carbon atoms and in some embodiments from 2 to 6 carbon atoms or 2 to 4 carbon atoms and containing at least one triple bond. The term “alkynyl” is also meant to include those hydrocarbyl groups having one triple bond and one double bond. For example, C₂₋₆ alkynyl includes ethynyl, propynyl, and the like.

“Phosphoramidate alkylating agent” refers to an alkylating agent comprising one or more Z⁵—X⁵—Y⁵ moieties bonded to an —O—P(Z1) moiety, where Z⁵ is a heteroatom such as nitrogen, sulfur or oxygen, X⁵ is optionally substituted ethylene, Y⁵ is halo or another leaving group, or Z⁵—X⁵—Y⁵ together form an aziridinyl (NCH₂CH₂) moiety, and Z¹ is defined as above. Such an alkylating agent can react with a DNA or another nucleic acid or protein. In some instances an alkylating agent can be cross-linked with DNA.

The term “optionally substituted” refers to a substituted or unsubstituted group. The group may be substituted with one or more substituents, such as e.g., 1, 2, 3, 4 or 5 substituents. Preferably, the substituents are selected from the group consisting of oxo, halo, —CN, NO₂, —N₂+, —CO₂R¹⁰⁰, —OR¹⁰⁰, —SR¹⁰⁰, —SOR¹⁰⁰, —SO₂R¹⁰⁰, —NR¹⁰⁰SO₂R¹⁰⁰, —NR¹⁰¹R¹⁰², —CONR¹⁰¹R¹⁰², —SO₂NR¹⁰¹R¹⁰², C₁-C₆ alkyl, C₁-C₆ alkoxy, —CR¹⁰⁰═C(R¹⁰⁰)₂, —CCR¹⁰⁰, C₃-C₁₀ cycloalkyl, C₃-C₁₀ heterocyclyl, C₆-C₁₂ aryl and C₂-C₁₂ heteroaryl, or a divalent substituent such as —O—(CH²)—O—, —O—(CH₂)₂—O—, and, 1-4 methyl substituted version thereof, wherein each R¹⁰⁰, R¹⁰¹, and R¹⁰² independently is hydrogen or C₁-C₈ alkyl; C₃-C₁₂ cycloalkyl; C₃-C₁₀ heterocyclyl; C₆-C₁₂ aryl; or C₂-C₁₂ heteroaryl; or R¹⁰⁰ and R¹⁰² together with the nitrogen atom to which they are attached to form a 5-7 membered heterocycle; wherein each alkyl, cycloalkyl, heterocyclyl, aryl, or heteroaryl is optionally substituted with 1-3 halo, 1-3 C₁-C₆ alkyl, 1-3 C₁-C₆ haloalkyl or 1-3 C₁-C₆ alkoxy groups. Preferably, the substituents are selected from the group consisting of chloro, fluoro, —OCH₃, methyl, ethyl, iso-propyl, cyclopropyl, —CO₂H and salts and C₁-C₆ alkyl esters thereof, CONMe₂, CONHMe, CONH₂, —SO₂Me, —SO₂NH₂, —SO₂NMe₂, —SO₂NHMe, —NHSO₂Me, —NHSO₂CF₃, —NHSO₂CH₂Cl, —NH₂, —OCF₃, —CF₃ and —OCHF₂.

“Alkylene” refers to divalent saturated aliphatic hydrocarbyl groups having from 1 to 10 carbon atoms and, in some embodiments, from 1 to 6 carbon atoms. “Cᵤ₋ᵥ alkylene” refers to alkylene groups having from u to v carbon atoms. The alkylidene and alkylene groups include branched and straight chain hydrocarbyl groups. For example, “C₁₋₆ alkylene” includes methylene, ethylene, propylene, 2-methylpropylene, pentylene, and the like.

“Heteroalkylene” refers to an alkylene wherein a chain carbon atom is replaced with a heteroatom such as O, S, N, or P, or a heteroatom containing substituent.

The “drugs” regarding D herein includes without limitation, gemcitibine, erlotinib, meturedepa, uredepa, altretamine, imatinib, triethylenemelamine, trimethylmelamine, chlorambucil, chlornaphazine, estramustine, gefitinib, mechlorethamine, mechlorethamine oxide hydrochloride, melphalan, novembichin, phenesterine, prednimustine, trofosfamide, uracil mustard, carmustine, chlorozotocin, fotemustine, nimustine, ranimustine, dacarbazine, mannomustine, mitobronitol, mitolactol, pipobroman, aclacinomycins, actinomycin, anthramycin, azaserine, bleomycin, cactinomycin, carubicin, carzinophilin, chromomycin, dactinomycin, daunorubicin, daunomycin, 6-diazo-5-oxo-1-norleucine, mycophenolic acid, nogalamycin, olivomycin, peplomycin, plicamycin, porfiromycin, puromycin, streptonigrin, streptozocin, tubercidin, ubenimex, zinostatin, zorubicin, denopterin, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, 5-fluorouracil, tegafur, L-asparaginase, pulmozyme, aceglatone, aldophosphamide glycoside, aminolevulinic acid, amsacrine, bestrabucil, bisantrene, defofamide, demecolcine, diaziquone, elfornithine, elliptinium acetate, etoglucid, flutamide, hydroxyurea, interferon-alpha, interferon-beta, interferon-gamma, interleukin-2, lentinan, mitoguazone, mitoxantrone, mopidamol, nitracrine, pentostatin, phenamet, pirarubicin, podophyllinic acid, 2-ethylhydrazide, procarbazine, razoxane, sizofiran, spirogermanium, paclitaxel, tamoxifen, erlotonib, teniposide, tenuazonic acid, triaziquone, 2,2′,2″-trichlorotriethylamine, urethan, vinblastine, and vincristine.

“Administering” or “administration of” a drug to a patient (and grammatical equivalents of this phrase) refers to direct administration, which may be administered to a patient by a medical professional or may be self-administered, and/or indirect administration, which may be the act of prescribing a drug. For example, a physician who instructs a patient to self-administer a drug and/or provides a patient with a prescription for a drug is administering the drug to the patient.

“Cancer” refers to leukemias, lymphomas, carcinomas, and other malignant tumors, including solid tumors, of potentially unlimited growth that can expand locally by invasion and spread throughout the bodyby metastasis. Examples of cancers include, but are not limited to, cancer of the adrenal gland, bone, brain, breast, bronchi, colon and/or rectum, gallbladder, head and neck, kidneys, larynx, liver, lung, neural tissue, pancreas, prostate, parathyroid, skin, stomach, and thyroid. Certain other examples of cancers include, acute and chronic lymphocytic and granulocytic tumors, adenocarcinoma, adenoma, basal cell carcinoma, poor cervical intraepithelial differentiation and in situ carcinoma, Ewing's sarcoma, epidermoid carcinomas, giant cell tumor, glioblastoma multiforma, hairy-cell tumor, intestinal ganglioneuroma, hyperplastic corneal nerve tumor, islet cell carcinoma, Kaposi's sarcoma, leiomyoma, leukemias, lymphomas, malignant carcinoid, malignant melanomas, malignant hypercalcemia, marfanoid habitus tumor, medullary epithelial carcinoma, metastatic skin carcinoma, mucosal neuroma, myeloma, mycosis fungoides, neuroblastoma, osteo sarcoma, osteogenic and other sarcoma, ovarian tumor, pheochromocytoma, polycythermia vera, primary brain tumor, small-cell lung tumor, squamous cell carcinoma of both ulcerating and papillary type, hyperplasia, seminoma, soft tissue sarcoma, retinoblastoma, rhabdomyosarcoma, renal cell tumor, topical skin lesion, reticulum cell sarcoma, and Wilm's tumor.

“Inflammation” is preferably the inflammation which causes pain due to the prostaglandin E2/F2 from the above-mentioned arachidonic acid-cyclooxygenase-prostaglandin pathway.

“Patient” and “subject” are used interchangeably to refer to a mammal in need of treatment for cancer. Generally, the patient is a human. Generally, the patient is a human diagnosed with cancer. In certain embodiments, a “patient” or “subject” may refer to a non-human mammal used in screening, characterizing, and evaluating drugs and therapies, such as, a non-human primate, a dog, cat, rabbit, pig, mouse or a rat.

“Prodrug” refers to a compound that, after administration, is metabolized or otherwise converted to a biologically active or more active compound (or drug) with respect to at least one property. A prodrug, relative to the drug, is modified chemically in a manner that renders it, relative to the drug, less active or inactive, but the chemical modification is such that the corresponding drug is generated by metabolic or other biological processes after the prodrug is administered. A prodrug may have, relative to the active drug, altered metabolic stability or transport characteristics, fewer side effects or lower toxicity, or improved flavor (for example, see the reference Nogrady, 1985, Medicinal Chemistry A Biochemical Approach, Oxford University Press, New York, pages 388-392, incorporated herein by reference). A prodrug may be synthesized using reactants other than the corresponding drug.

“Solid tumor” refers to solid tumors including, but not limited to, metastatic tumors in bone, brain, liver, lungs, lymph node, pancreas, prostate, skin and soft tissue (sarcoma).

“Therapeutically effective amount” of a drug refers to an amount of a drug that, when administered to a patient with cancer, will have the intended therapeutic effect, e.g., alleviation, amelioration, palliation or elimination of one or more manifestations of cancer in the patient. A therapeutic effect does not necessarily occur by administration of one dose, and may occur only after administration of a series of doses. Thus, a therapeutically effective amount may be administered in one or more administrations.

“Treatment of” a condition or patient refers to taking steps to obtain beneficial or desired results, including clinical results. For purposes of this invention, beneficial or desired clinical results include, but are not limited to, alleviation or improvement of one or more symptoms of cancer; diminishment of extent of disease; delay or slowing of disease progression; alleviation, palliation, or stabilization of the disease state; or other beneficial results. Treatment of cancer may, in some cases, result in partial response or stable disease.

“Tumor cells” refers to tumor cells of any appropriate species, e.g., mammalian such as murine, canine, feline, equine or human.

The above description of embodiments of the present invention does not limit the present invention. Those skilled in the art can make various modifications and changes according to the present invention, and any modification and change within the spirit of the present invention shall be covered in the scope of the claims appended to the present invention.

An in vitro experiment and an in vivo experiment in cynomolgus monkeys are provided below to verify the analgesic effects of the compounds.

### In Vitro Experiments

Experimental apparatus: Waters Acquity I Class UPLC Ultra Performance Liquid Chromatograph equipped with a Xevo G2-XS Q Tof HRMS Quadrupole Time-of-flight High-Resolution Mass Spectrometer

Buffers and Materials:

1. PBS phosphate buffered saline solution,

2. PBS phosphate buffered saline solution of 20 mM NADPH

3. PBS phosphate buffered saline solution of 250 μg/mL AKR1C3

4. 50% MeOH/H2O solution of 250 μM AST-3424

5. 50% MeOH/H2O solution of 250 μM progesterone

6. 100% acetonitrile solution of 1 μg/mL propranolol

### Experimental Procedures

In step 1, the reaction mixtures were prepared into Eppendorf tubes in quadruplicate (n=4) according to the table below and mixed gently.

| | Negative | |
|---|---|---|
| Materials | controls (μL) | Samples (μL) |
| PBS | 68 | 58 |
| NADPH (20 mM) | 10 | 10 |
| AKR1C3 (250 μg/mL) | 10 | 10 |
| AST-3424 (250 μM) | 0 | 10 |

In step 2, the above mixtures were pre-incubated in duplicate at 37° C. for 30 minutes and 60 minutes.

In step 3, another 10 μL of PBS phosphate buffered saline solution of 20 mM NADPH and 2 μL of 50% MeOH/H₂O solution of 250 μM progesterone were added to each Eppendorf tube and mixed gently.

In step 4, 50 μL of the mixture in the above step was immediately transferred to 100 μL of 100% acetonitrile solution of 1 μg/mL propranolol (internal standard (IS)).

In step 5, the remaining samples were incubated at 37° C. for 30 minutes, and 100 μL of 100% acetonitrile solution of 1 μg/mL propranolol (internal standard (IS)) was added.

In step 6, 100 μL of reagent water was added to all the samples, vortexed at 1,100 rpm for 5 minutes, and centrifuged at 15,000 rpm for 10 minutes at room temperature.

In step 7, all the samples were loaded on LC/MS to determine the content of reduced progesterone, namely 20α-dihydroprogesterone.

The test conditions for the LC-MS apparatus are shown below

| Items | Conditions |
|---|---|
| Apparatus: | Waters Acquity I Class Liquid Chromatograph |
| Chromatographic | Acquity UPLC BEHC18 Chromatographic Column |
| column: | (50*2.1 mm, 1.7 μm) |
| Flow rate: | 0.4 mL/min |
| Injection volume: | 3 μL |
| The composition of | A: 0.1% (V/V) formic acid aqueous solution |
| the mobile phase: | B: 0.1% (V/V) formic acid acetonitrile solution |
| The temperature of | 40° C. |
| the column oven: | |
| Detector: | Quadrupole Time-of-flight Mass Spectrometer |
| | Q-TOF MS |

The Gradient of Elution of the Liquid Phase

| Time (min) | A (%) | B (%) |
|---|---|---|
| 0.00 | 90.0 | 0.0 |
| 1.5 | 5.0 | 95.0 |
| 2.00 | 5.0 | 95.0 |
| 2.30 | 90.0 | 10.0 |
| 3.00 | 90.0 | 10.0 |

Parameters of the Quadrupole Time-of-Flight Mass Spectrometer

| Items | Parameters |
|---|---|
| Capillary voltage (kV) | 2.5 |
| Cone voltage (V) | 40 |
| Source temperature (° C.) | 100 |
| Flow rate of cone gas (L/h) | 50 |
| Flow rate of desolvation gas | 600 |
| (L/h) | |
| Ionization mode (Interface | ES, Positive |
| type) | |
| Analyser mode | Sensitivity |
| Scan range | 50-800 m/z |

In step 9, the reduced progesterone (20α-dihydroprogesterone) is calculated: the peak area of the reduced progesterone, namely 20α-dihydroprogesterone and propranolol, in each sample was determined by LC/MS. The peak area ratios of reduced progesterone to propranolol (i.e., the ratios in the above table) were calculated, and when the time is 0 the ratio is set to 0%.

AKR1C3 activity (%)=[(the amount of reduced progesterone after normalization of the sample) 30 min−(the amount of reduced progesterone after normalization of the sample) 0 min]/[(the amount of reduced progesterone after normalization of the negative control group) 30 min−(the amount of reduced progesterone after normalization of the negative control group) 0 min]*100.

The AKR1C3 activity results in the above table were calculated according to the above formula.

### Experimental Results

| | | | The peak areas of the liquid phase | | | | | | | | |
|---|---|---|---|---|---|---|---|---|---|---|---|
| | Duration | | | Progesterone | | Reduced progesterone | | | Propranolol | | AKR1C3 |
| | Incubation | Reaction | AST-3424 | Values | Averages | Values | Averages | Ratios* | Values | Averages | activity % |
| Negative | 30 min | 0 | Absent | 36658 | 36835 | 150 | 158 | 0.0034 | 51141 | 46879 | 0 |
| control | | | | 37011 | | 166 | | | 42616 | | |
| groups | 60 min | | | 35244 | 35125 | 87 | 83 | 0.0019 | 42794 | 43650 | 0 |
| | | | | 35006 | | 79 | | | 44506 | | |
| | 30 min | 30 min | | 28675 | 26228 | 5616 | 6617 | 0.1506 | 43589 | 43935 | 100 |
| | | | | 23780 | | 7617 | | | 44280 | | |
| | 60 min | | | 25917 | 26492 | 6747 | 6051 | 0.1343 | 44258 | 45067 | 100 |
| | | | | 27067 | | 5354 | | | 45876 | | |
| Samples | 30 min | 0 | 154017 | 37107 | 37438 | 70 | 72 | 0.0017 | 43247 | 43436 | 0 |
| | | | 154843 | 37769 | | 74 | | | 43625 | | |
| | 60 min | | 108351 | 38050 | 37989 | 58 | 64 | 0.0015 | 43907 | 43697 | 0 |
| | | | 107508 | 37928 | | 70 | | | 43487 | | |
| | 30 min | 30 min | 104748 | 35187 | 35210 | 344 | 331 | 0.0074 | 45657 | 45051 | 3.9 |
| | | | 110630 | 35234 | | 319 | | | 44445 | | |
| | 60 min | | 52296 | 32636 | 32520 | 622 | 616 | 0.0137 | 45395 | 45101 | 9.2 |
| | | | 51309 | 32404 | | 610 | | | 44807 | | |

### Analysis and Summary of the Experimental Results

Table: The effect of AST-3424 on AKR1C3 activity

% AKR1C3 activity

| The time of | 0 μM | 5 μM |
|---|---|---|
| incubation (min) | AST-3424 | AST-3424 |
| 30 | 100% | 3.9% |
| 60 | 100% | 9.2% |

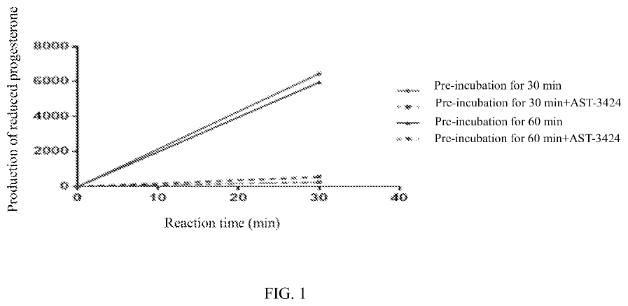

The effect of AST-3424 on the production of reduced progesterone is shown in FIG. 1.

The aforementioned in vitro experiment proves that after pre-incubation for 30 minutes and 60 minutes, AST-3424 at a concentration of 5 μM basically inhibited AKR1C3 activity: compared with the negative control groups, the production of the reduced progesterone, namely 20α-dihydroprogesterone, was reduced to 3.9% and 9.2%, respectively, proving that the compound AST-3424 and the similar compounds disclosed in the patent applications No. PCT/US2016/021581, PCT/US2016/025665 and PCT/US2016/062114 are inhibitors of AKR1C3 enzyme.

### II. In Vivo Experiment

An experiment with three cynomolgus monkeys was performed according to the table below.

| | | Administration | | | | | |
|---|---|---|---|---|---|---|---|
| | | | | The | | | |
| | | | | concentration of | | | |
| | | | | the solution of | The | | |
| | | | The dosage of the | the test | volume of | | |
| | Amount | Test | test substance | substance | administration | Mode of | Collected |
| Group | Male | substance | (mg/kg) | (mg/mL) | (mL/kg) | administration | Sample |
| 1 | 3 | AST3424 | 1/Compound | 0.2 | 5 | Intravenous | Plasma/ |
| | | | | | | infusion | serum |

Four male cynomolgus monkeys were purchased from Guangxi Xiongsen Primate Development and Experiment Co., Ltd., and all of them were healthy cynomolgus monkeys that had passed the physical examination and have no abnormalities. Among them, three cynomolgus monkeys were used in the experiment of administration and the remaining one was used for preparing blank plasma.

Before administration, and 6, 24, 48 and 72 hours after the start of administration, 1 mL of blood was collected from the femoral vein or other suitable vein, and placed in a blood collection tube without anticoagulant. After collected, the blood sample was placed on ice and allowed to stand for 30-60 minutes before being centrifuged at 3,500 rpm for 10 minutes at 2-8° C. to separate the serum. The collected serum was stored at −80° C. before being analyzed.

Prostaglandins E2 and F2 in serum samples were analyzed by conventional ELISA methods. The measurement results are as follows.

The concentrations of prostaglandins E2 and F2 in the serum after single administration to cynomolgus monkeys by intravenous infusion

| | The measured concentration of prostaglandin F2 (pg/ml) | | | | |
|---|---|---|---|---|---|
| Time point | 101 | 102 | 103 | Mean | SD |
| Before | 1828.51 | 310.17 | 1125.58 | 1088.09 | 759.86 |
| administration | | | | | |
| 6 h | 165.32 | 260.80 | 300.65 | 242.26 | 69.54 |
| 24 h | 816.63 | 3460.25 | 4208.19 | 2828.36 | 1781.89 |
| 48 h | 183.73 | 216.12 | 541.05 | 313.63 | 197.61 |
| 72 h | 441.76 | 968.01 | 1369.67 | 926.48 | 465.35 |
| | The measured concentration of prostaglandin E2 (pg/ml) | | | | |
| Time point | 101 | 102 | 103 | Mean | SD |
| Before | 713.91 | 233.63 | 461.26 | 469.60 | 240.25 |
| administration | | | | | |
| 6 h | 210.67 | 214.73 | 670.73 | 365.38 | 264.45 |
| 24 h | 450.48 | 822.21 | 2735.20 | 1335.97 | 1225.95 |
| 48 h | 322.13 | 168.84 | 276.35 | 255.78 | 78.69 |
| 72 h | 408.15 | 521.78 | 663.03 | 530.99 | 127.69 |

After 0.58 mg/kg of AST3424 was administered to cynomolgus monkeys, both prostaglandins E2 and F2 decreased; they fluctuatingly increased at 24 h, which was speculated to be related to the time characteristics of the secretion of prostaglandins E2 and F2 by the cynomolgus monkeys themselves. This demonstrates that AST3424 can inhibit the secretion of prostaglandins E2 and F2 by cynomolgus monkeys.

The causes of pain directly arising from a tumor and bone infiltration and metastasis include the following: direct bone involvement and direct activation of local nociceptors; compression of adjacent nerves, blood vessels and soft tissues by the tumor; and release of PGE1 and PGF2 from bone infiltration by the tumor. PGF2 is a strong pain-causing factor. The aforementioned animal experiment shows that the AST-3424 compound can greatly reduce the content of PGF2, thereby achieving the effect of treating/alleviating the pain caused by cancer or tumor.

### III. Experiment Conclusion

The above in vitro and in vivo experiments verify that the aforementioned specific substrate, namely DNA alkylating agent targeting overexpressed aldo-keto reductase AKR1C3, can inhibit the activity of the aldo-keto reductase AKR1C3. Moreover, the animal experiment proves that the specific substrates can reduce the content of prostaglandin E2/F2 in the blood. Accordingly, it can be proved that the aforementioned compound AST-3424 and the similar compounds disclosed in the patent applications No. PCT/US2016/021581, PCT/US2016/025665 and PCT/US2016/062114 are inhibitors of AKR1C3 enzyme, which can block the production of prostaglandin E2/F2, reduce its content, and has an analgesic effect.

1. A method of preventing, treating or alleviating pain in a subject comprising the step of administering to the subject in need thereof an effective amount of a compound of formula I or a pharmaceutically acceptable salt, or a solvate thereof, 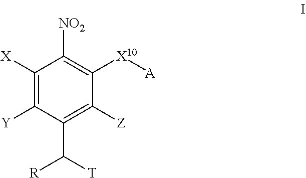
wherein
X¹⁰ is O, S, SO, or SO₂;
A is C₆-C₁₀ aryl or substituted aryl, 5-15 membered heteroaryl or substituted heteroaryl, or —N═CR¹R², wherein each R¹ and R² independently is hydrogen, C₁-C₆ alkyl, C₃-C₈ cycloalkyl, C₆-C₁₀ aryl, 4-15 membered heterocycle, 5-15 membered heteroaryl, ether, —CONR¹³R¹⁴, or —NR¹³COR¹⁴;
each X, Y, and Z independently is hydrogen, CN, halo, C₁-C₆ alkyl, C₂-C₆ alkenyl, C₂-C₆ alkynyl, C₃-C₈ cycloalkyl, C₆-C₁₀ aryl, 4-15 membered heterocycle, 5-15 membered heteroaryl, ether, —CONR¹³R¹⁴, or —NR¹³COR¹⁴;
R is hydrogen, C₁-C₆ alkyl, C₂-C₆ alkenyl, C₂-C₆ alkynyl, C₃-C₈ cycloalkyl, C₆-C₁₀ aryl, 4-15 membered heterocycle, 5-15 membered heteroaryl, ether, —CONR¹³R¹⁴, or —NR¹³COR¹⁴;
each R¹³ and R¹⁴ independently is hydrogen, C₁-C₆ alkyl, C₃-C₈ cycloalkyl, C₆-C₁₀ aryl, 4-15 membered heterocycle, 5-15 membered heteroaryl or ether, or R¹³ and R¹⁴ together with the nitrogen atom to which they are bonded to form 5-7 membered heterocyclyl group;
T comprises a phosphoramidate alkylating agent; and
wherein the alkyl, alkenyl, alkynyl, cycloalkyl, aryl, heterocycle, heteroaryl, and ether groups are optionally substituted,
thereby preventing, treating or alleviating pain in the subject.

2. The method according to claim 1, wherein in the compound of formula I, T is OP(Z¹)(NR³⁰CH₂CH₂X¹)₂, OP(Z¹)(NR³⁰₂)(N(CH₂CH₂X¹)₂), OP(Z¹)(N(CH₂)₂)₂ or OP(Z¹)(N(CH₂CH₂X¹)₂)₂, wherein each R³⁰ independently is hydrogen or C₁-C₆ alkyl or two R³⁰ groups together with the nitrogen atom to which they are bonded to form 5-7 membered heterocyclyl group, Z¹ is O or S, and X¹ is Cl, Br, or OMs or other leaving groups.

3. The method according to claim 1, wherein in the compound of formula I, T is OP(Z¹)(NHCH₂CH₂Cl)₂, OP(Z¹)(NHCH₂CH₂Br)₂, OP(Z¹)(NH₂)(N(CH₂CH₂X¹)₂), OP(Z¹)(N(CH₂)₂)₂ or OP(Z¹)(N(CH₂CH₂Cl)₂)₂, wherein Z¹ is O or S, and X¹ is Cl, Br, or OMs.

4. The method according to claim 1, wherein in the compound of formula I, T is OP(O)(N(CH₂CH₂))₂, OP(O)(NHCH₂CH₂Cl)₂, OP(O)(NHCH₂CH₂Br)₂ or OP(O)(NH₂)(N(CH₂CH₂Cl)₂).

5. The method according to claim 1, wherein the compound of formula I is

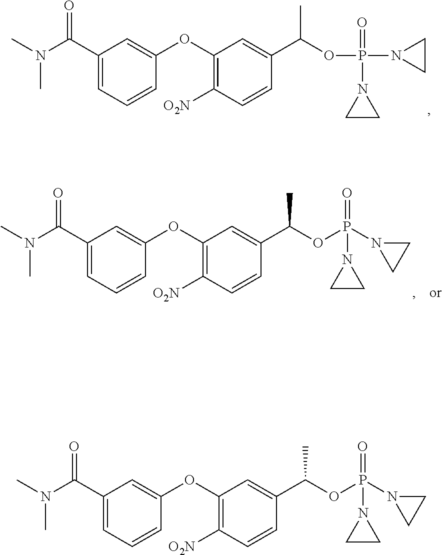 [SMILES: CC(OP(=O)(N1CC1)N1CC1)c1ccc([N+](=O)[O-])c(Oc2cccc(C(=O)N(C)C)c2)c1 ; C[C@@H](OP(=O)(N1CC1)N1CC1)c1ccc([N+](=O)[O-])c(Oc2cccc(C(=O)N(C)C)c2)c1 ; C[C@H](OP(=O)(N1CC1)N1CC1)c1ccc([N+](=O)[O-])c(Oc2cccc(C(=O)N(C)C)c2)c1]

6. A method of preventing, treating or alleviating pain in a subject comprising the step of administering to the subject in need thereof an effective amount of a compound of formula II or a pharmaceutically acceptable salt, or a solvate thereof, 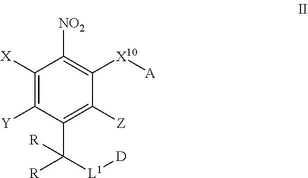
wherein
X¹⁰ is O, S, SO, or SO₂;
A is C₆-C₁₀ aryl or substituted aryl, 5-15 membered heteroaryl or substituted heteroaryl, or —N═CR¹R², wherein each R¹ and R² independently is hydrogen, C₁-C₆ alkyl, C₃-C₈ cycloalkyl, C₆-C₁₀ aryl, 4-15 membered heterocycle, 5-15 membered heteroaryl, ether, —CONR¹³R¹⁴ or —NR¹³COR¹⁴;
each X, Y, and Z independently is hydrogen, CN, halo, C₁-C₆ alkyl, C₂-C₆ alkenyl, C₂-C₆ alkynyl, C₃-C₈ cycloalkyl, C₆-C₁₀ aryl, 4-15 membered heterocycle, 5-15 membered heteroaryl, ether, —CONR¹³R¹⁴, or —NR¹³COR¹⁴;
each R independently is hydrogen, C₁-C₆ alkyl, C₂-C₆ alkenyl, C₂-C₆ alkynyl, C₃-C₈ cycloalkyl, C₆-C₁₀ aryl, 4-15 membered heterocycle, 5-15 membered heteroaryl, ether, —CONR¹³R¹⁴, or —NR¹³COR¹⁴;
each R¹³ and R¹⁴ independently is hydrogen, C₁-C₆ alkyl, C₃-C₈ cycloalkyl, C₆-C₁₀ aryl, 4-15 membered heterocycle, 5-15 membered heteroaryl, or ether, or R¹³ and R¹⁴ together with the nitrogen atom to which they are bonded to form 5-7 membered heterocyclyl group;
L¹ is selected from the group consisting of: 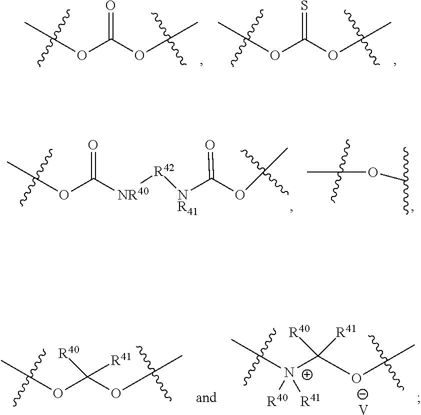
wherein R⁴⁰ and R⁴¹ are independently hydrogen, C₁-C₆ alkyl, C₂-C₆ alkenyl, C₂-C₆ alkynyl, C₃-C₈ cycloalkyl, C₆-C₁₀ aryl, 4-15 membered heterocycle, or 5-15 membered heteroaryl;
R⁴² is C₂-C₃ alkylene or heteroalkylene optionally substituted with 1-3 C₁-C₆ alkyl; V(−) is any anion, preferably, a pharmaceutically acceptable anion; and
D is a moiety such that D-OH is an anticancer drug wherein OH is an aliphatic or a phenolic hydroxy group or is an OH moiety attached to a phosphorous atom as provided herein; in other words, D is the remaining group in the anticancer drug D-OH after the hydroxyl group is removed therefrom;
alternatively
L¹ is: 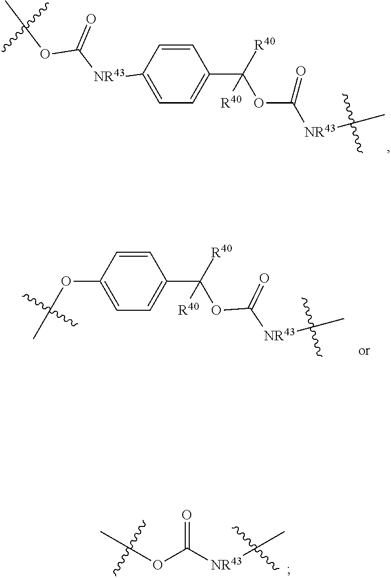
wherein R⁴⁰ is defined as above, R⁴³ is hydrogen or together with D forms a heterocycle, and the phenyl moiety is optionally substituted, and
D is a moiety such that D-NR⁴³H is an anticancer drug; in other words, D is the remaining group in the anticancer drug D-NR⁴³H after amino or amine is removed therefrom;
alternatively
L¹ is a bond, —O—C(R⁴⁰R⁴¹)₂—, —O—C(R⁴⁰R⁴¹)—NR⁴⁰R⁴¹(+)—C(R⁴⁰R⁴¹)— or 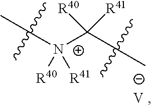
wherein R⁴⁰, R⁴¹ and V are defined as above, and
D is an anticancer drug containing a primary or a secondary amine, wherein the primary or the secondary amine is bonded to L¹; and
wherein the alkyl, alkenyl, alkynyl, cycloalkyl, aryl, heterocycle, heteroaryl, and ether groups are optionally substituted,
thereby preventing, treating or alleviating pain in the subject.

7. The method according to claim 6, wherein the compound of formula II is a compound of formula II-A, 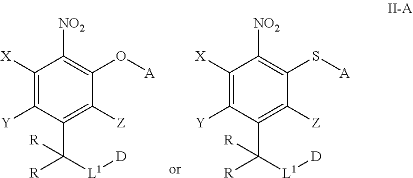
wherein the remaining variables are as defined in claim 6.

8. The method according to claim 6, wherein the compound of formula II is a compound of formula IIA-1: 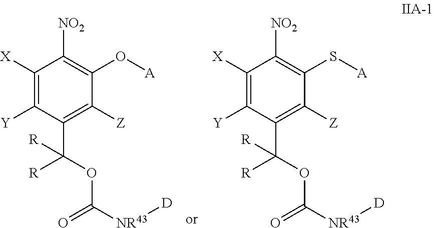
wherein the remaining variables are as defined in claim 6, and D is a moiety of the cytotoxic agent HNR⁴³-D containing a primary or a secondary amine group; in other words, D is the remaining group in the cytotoxic agent HNR⁴³-D after the primary or a secondary amine group is removed therefrom;
alternatively,
the compound is of formula III-2 or IIIA-3: 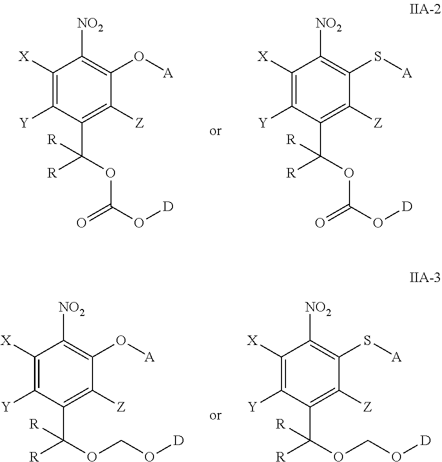
wherein the remaining variables are as defined in claim 6, and D is a moiety of the cytotoxic agent HO-D containing at least one hydroxyl group; in other words, D is the remaining group in the cytotoxic agent HO-D after the hydroxyl group is removed therefrom;
alternatively,
the compound is of formula IIA-4, IIA-6 or IIA-6-i: 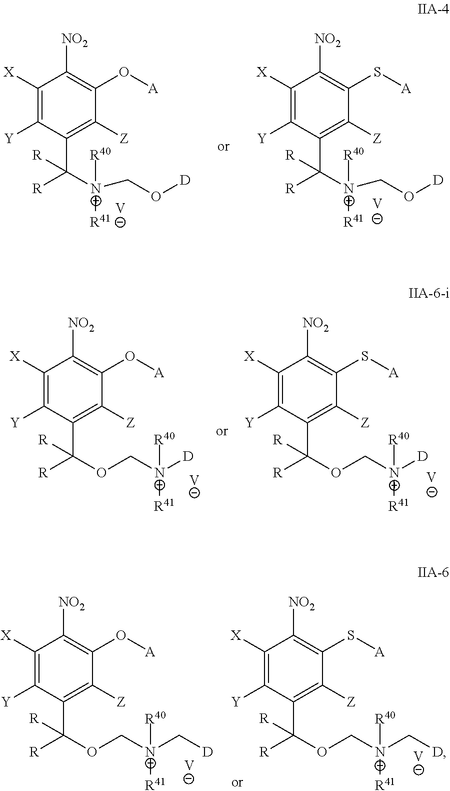
wherein the variables are as defined in claim 6; wherein in IIA-4, HO-D is cytotoxic agent containing at least one hydroxyl group; in other words, D is a moiety of the cytotoxic agent HO-D containing at least one hydroxyl group, or D is the remaining group in the cytotoxic agent HO-D after the hydroxyl group(s) is/are removed therefrom; in IIA-6-i, DNR⁴⁰R⁴¹ is a drug; in other words, D is a moiety such that DNR⁴⁰R⁴¹ is an anticancer drug or D is the remaining moiety in the anticancer DNR40R⁴¹ after NR⁴⁰R⁴¹ is removed therefrom; and in IIA-6, D is a drug containing a secondary amine, wherein the secondary amine is bonded to the methylene group; in other words, D is a drug containing a secondary amine, and is bonded via the secondary amine contained therein to the methylene group so as to be linked to —NR⁴⁰R⁴¹ as shown in the above formula IIA-4, IIA-6 or IIA-6-I;
alternatively,
the compound is of IIA-5 or IIA-7: 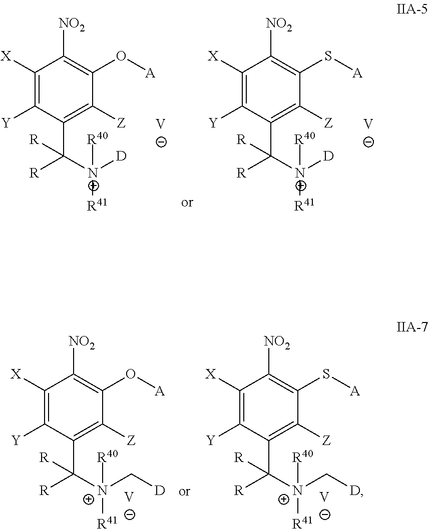
wherein the variables are as defined in claim 6; wherein in IIA-5, DNR⁴⁰R⁴¹ is a drug; in other words, D is a moiety such that DNR⁴⁰R⁴¹ is an anticancer drug or D is the remaining moiety in the anticancer DNR40R⁴¹ after NR⁴⁰R⁴¹ is removed therefrom; and IIA-7, D is a drug containing a secondary amine, wherein the secondary amine is bonded to the methylene group as shown in the above formula IIA-5 or IIA-7.

9. The method according to claim 6, wherein in the compound of formula II, Z is hydrogen, or X is hydrogen, or Y is hydrogen/halo.

10. The method according to claim 6, wherein in the compound of formula II, A is substituted or unsubstituted C₆-C₁₀ aryl, or phenyl, or 5-15 membered heteroaryl, or pyridyl, or —N═CR¹R², wherein R¹ and R² are as defined in claim 6.

11. The method according to claim 6, wherein in the compound of formula II, each R is hydrogen, or one of the R groups is hydrogen and the other R group is C₁-C₆ alkyl, or both R groups are non-hydrogen substituents as defined in claim 6, or R is methyl.

12. The method according to claim 6, wherein in the compound of formula II, each of R⁴⁰, R⁴¹ and R⁴³ is independently hydrogen or methyl and R⁴² is —CH₂—CH₂— or —CH₂—C(Me)₂-.

13. The method according to claim 6, wherein in the compound of formula II, D excludes a phosphoramidate alkylating agent such as —P(Z¹)(NR³⁰CH₂CH₂X¹)₂, —P(Z¹)(NR³⁰₂)(N(CH₂CH₂X¹)₂), —P(Z¹)(N(CH₂CH₂))₂ or —P(Z¹)(N(CH₂CH₂X¹)₂)₂, wherein each R³⁰ independently is hydrogen or C₁-C₆ alkyl or 2R³⁰s together with the nitrogen atom to which they are bonded to form 5-7 membered heterocyclyl group, Z¹ is O or S, and X¹ is Cl, Br, or OMs or another leaving group.

14. The method according to claim 1, wherein the pharmaceutically acceptable salt is a basic salt or an acid salt or the solvate is hydrate or alcoholate; or the pain is a pain caused by cancer or inflammation.

15-16. (canceled)

17. A pharmaceutical composition comprising the compound of formula I 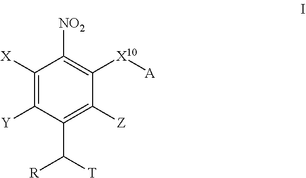
wherein
X¹⁰ is O, S, SO, or SO₂;
A is C₆-C₁₀ aryl or substituted aryl, 5-15 membered heteroaryl or substituted heteroaryl, or —N═CR¹R², wherein each R¹ and R² independently is hydrogen, C₁-C₆ alkyl, C₃-C₈ cycloalkyl, C₆-C₁₀ aryl, 4-15 membered heterocycle, 5-15 membered heteroaryl, ether, —CONR¹³R¹⁴, or —NR¹³COR¹⁴;
each X, Y, and Z independently is hydrogen, CN, halo, C₁-C₆ alkyl, C₂-C₆ alkenyl, C₂-C₆ alkynyl, C₃-C₈ cycloalkyl, C₆-C₁₀ aryl, 4-15 membered heterocycle, 5-15 membered heteroaryl, ether, —CONR¹³R¹⁴, or —NR¹³COR¹⁴;
R is hydrogen, C₁-C₆ alkyl, C₂-C₆ alkenyl, C₂-C₆ alkynyl, C₃-C₈ cycloalkyl, C₆-C₁₀ aryl, 4-15 membered heterocycle, 5-15 membered heteroaryl, ether, —CONR¹³R¹⁴, or —NR¹³COR¹⁴;
each R¹³ and R¹⁴ independently is hydrogen, C₁-C₆ alkyl, C₃-C₈ cycloalkyl, C₆-C₁₀ aryl, 4-15 membered heterocycle, 5-15 membered heteroaryl or ether, or R¹³ and R¹⁴ together with the nitrogen atom to which they are bonded to form 5-7 membered heterocyclyl group;
T comprises a phosphoramidate alkylating agent; and
wherein the alkyl, alkenyl, alkynyl, cycloalkyl, aryl, heterocycle, heteroaryl, and ether groups are optionally substituted,
and a pharmaceutically acceptable auxiliary or excipient, for prevention, treatment or alleviation of pain caused by cancer or inflammation.

18. A method for treatment of pain caused by cancer or inflammation, the method comprising a step of administering the pharmaceutical composition of claim 17 to a subject in need of such treatment, thereby treating the pain in the subject; and a step for measuring the content of AKR1C3 reductase of cancer cells in the subject using AKR1C3 antibodies, where the content of AKR1C3 reductase is measured to be equal to or greater than the predetermined value.

19. The method according to claim 6, wherein the salt is a basic salt or an acid salt; or the solvate is hydrate or alcoholate; or the pain is a pain caused by cancer or inflammation.

20. A pharmaceutical composition comprising the compound of formula II 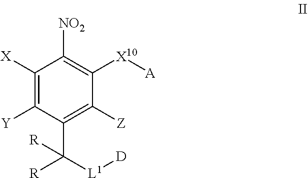
wherein
X¹⁰ is O, S, SO, or SO₂;
A is C₆-C₁₀ aryl or substituted aryl, 5-15 membered heteroaryl or substituted heteroaryl, or —N═CR¹R², wherein each R¹ and R² independently is hydrogen, C₁-C₆ alkyl, C₃-C₈ cycloalkyl, C₆-C₁₀ aryl, 4-15 membered heterocycle, 5-15 membered heteroaryl, ether, —CONR¹³R¹⁴ or —NR¹³COR¹⁴;
each X, Y, and Z independently is hydrogen, CN, halo, C₁-C₆ alkyl, C₂-C₆ alkenyl, C₂-C₆ alkynyl, C₃-C₈ cycloalkyl, C₆-C₁₀ aryl, 4-15 membered heterocycle, 5-15 membered heteroaryl, ether, —CONR¹³R¹⁴, or —NR¹³COR¹⁴;
each R independently is hydrogen, C₁-C₆ alkyl, C₂-C₆ alkenyl, C₂-C₆ alkynyl, C₃-C₈ cycloalkyl, C₆-C₁₀ aryl, 4-15 membered heterocycle, 5-15 membered heteroaryl, ether, —CONR¹³R¹⁴, or —NR¹³COR¹⁴;
each R¹³ and R¹⁴ independently is hydrogen, C₁-C₆ alkyl, C₃-C₈ cycloalkyl, C₆-C₁₀ aryl, 4-15 membered heterocycle, 5-15 membered heteroaryl, or ether, or R¹³ and R¹⁴ together with the nitrogen atom to which they are bonded to form 5-7 membered heterocyclyl group;
L¹ is selected from the group consisting of: 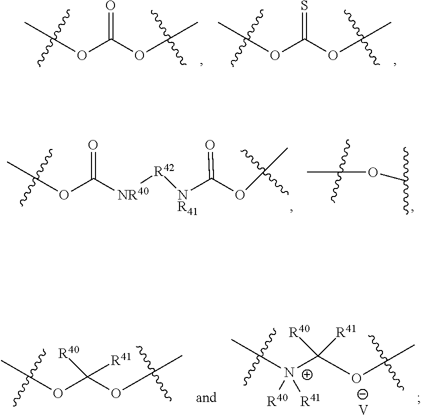
wherein R⁴⁰ and R⁴¹ are independently hydrogen, C₁-C₆ alkyl, C₂-C₆ alkenyl, C₂-C₆ alkynyl, C₃-C₈ cycloalkyl, C₆-C₁₀ aryl, 4-15 membered heterocycle, or 5-15 membered heteroaryl;
R⁴² is C₂-C₃ alkylene or heteroalkylene optionally substituted with 1-3 C₁-C₆ alkyl; V(−) is any anion, preferably, a pharmaceutically acceptable anion; and
D is a moiety such that D-OH is an anticancer drug wherein OH is an aliphatic or a phenolic hydroxy group or is an OH moiety attached to a phosphorous atom as provided herein; in other words, D is the remaining group in the anticancer drug D-OH after the hydroxyl group is removed therefrom;
alternatively
L1 is: 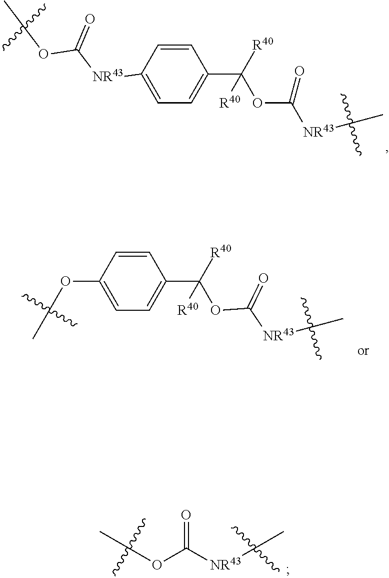
wherein R⁴⁰ is defined as above, R⁴³ is hydrogen or together with D forms a heterocycle, and the phenyl moiety is optionally substituted, and
D is a moiety such that D-NR⁴³H is an anticancer drug; in other words, D is the remaining group in the anticancer drug D-NR⁴³H after amino or amine is removed therefrom;
alternatively
L1 is a bond, —O—C(R⁴⁰R⁴¹)₂—, —O—C(R⁴⁰R⁴¹)—NR⁴⁰R⁴¹(+)—C(R⁴⁰R⁴¹)— or 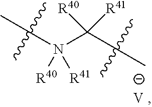
wherein R⁴⁰, R⁴¹ and V are defined as above, and
D is an anticancer drug containing a primary or a secondary amine, wherein the primary or the secondary amine is bonded to L¹; and
wherein the alkyl, alkenyl, alkynyl, cycloalkyl, aryl, heterocycle, heteroaryl, and ether groups are optionally substituted,
and a pharmaceutically acceptable auxiliary or excipient, for prevention, treatment or alleviation of pain caused by cancer or inflammation.

21. A method for treatment of pain caused by cancer or inflammation, the method comprising a step of administering the pharmaceutical composition of claim 20 to a subject in need of such treatment, thereby treating the pain in the subject; and a step for measuring the content of AKR1C3 reductase of cancer cells in a subject using AKR1C3 antibodies, where the content of AKR1C3 reductase is measured to be equal to or greater than the predetermined value.
